(19) United States
(12) Patent Application Publication
(10) Pub. No.: US 2021/0259669 A1
(43) Pub. Date: Aug. 26, 2021

(54) TELESCOPIC SHAFT FOR SURGICAL DEVICE

(71) Applicant: Covidien LP, Mansfield, MA (US)

(72) Inventors: Douglas M. Pattison, East Hartford, CT (US); Kevin S. Sniffin, Roxbury, CT (US)

(21) Appl. No.: 17/132,959

(22) Filed: Dec. 23, 2020

Related U.S. Application Data
(60) Provisional application No. 62/980,549, filed on Feb. 24, 2020.

(51) Int. Cl. A61B 17/00

(52) U.S. Cl. CPC A61B 17/00; A61B 34/30; A61B 2017/00991; A61B 2017/0046

(57) ABSTRACT

An elongate assembly for use with an endoscopic surgical device including manual, mechanical and/or electromechanical surgical tack appliers, surgical clip appliers, surgical staplers, surgical stitching devices and the like, has an elongate assembly including a selectively adjustable telescopic shaft. The telescopic shaft is transitionable between an extended configuration and a retracted configuration, in which, the entire telescopic shaft is disposed within a handle assembly of the endoscopic surgical device.

### CROSS-REFERENCE TO RELATED APPLICATIONS

This application claims the benefit of and priority to U.S. Provisional Patent Application Ser. No. 62/980,549 filed Feb. 24, 2020, the entire disclosure of which is incorporated by reference herein.

### TECHNICAL FIELD

This disclosure relates to surgical devices for performing endoscopic surgical procedures. More specifically, this disclosure relates to a selectively adjustable elongate assembly configured for use with the surgical devices.

### BACKGROUND

Hernias are abnormal protrusions of an organ or other body structure through a defect or natural opening in a covering membrane, muscle or bone. During surgical hernia repair procedures, surgical mesh materials are placed over the defect in the abdominal wall and are often sutured within the surgical opening by conventional suturing techniques. Traditionally, hernia repairs involved major invasive surgical procedures which often caused excessive trauma to the patient and necessitated long postoperative recuperative periods.

Recently, the use of laparoscopic and endoscopic surgical procedures has been relatively popular. During laparoscopic or endoscopic surgical procedures, access to a surgical site is achieved through a small incision or through a narrow cannula inserted through a small entrance wound in a patient.

### SUMMARY

In accordance with this disclosure, a surgical device includes a handle assembly, an elongate assembly detachably coupled to the handle assembly, and a cartridge operatively coupled to the handle assembly. The elongate assembly includes a telescopic shaft transitionable between a retracted configuration and an extended configuration to selectively adjust a length of the surgical device, and an adapter portion coupled to the telescopic shaft. The cartridge includes a plurality of anchors that are released from the cartridge when the handle assembly is actuated.

In an aspect, the telescopic shaft may be tapered along a length thereof.

In another aspect, the handle assembly may define an opening configured to receive the telescopic shaft therethrough.

In yet another aspect, the entire telescopic shaft may be received in the handle assembly when the telescopic shaft is in the retracted configuration.

In still yet another aspect, the telescopic shaft may define a lumen therethrough.

In still yet another aspect, the adapter portion may be distal of the telescopic shaft.

In an aspect, the adapter portion may have a diameter larger than a diameter of a distal portion of the telescopic shaft.

In another aspect, at least a portion of the adapter portion may be received in the handle assembly when the telescopic shaft is in the retracted configuration.

In another aspect, the telescopic shaft may include a plurality of segments concentrically arranged.

In yet another aspect, each segment of the plurality of segments may partially overlap adjacent segments.

In still yet another aspect, each segment may frictionally engage adjacent segments to maintain a relative position therewith.

In still yet another aspect, at least one of the telescopic shaft or the adapter portion may be formed of a biocompatible material.

In an aspect, the at least one of the telescopic shaft or the adapter portion may be formed of a medical grade metal.

In an aspect, the adapter portion of the elongate assembly may be configured to support the cartridge therein.

In another aspect, a length of the telescopic shaft in the extended configuration may be more than twice the length of the telescopic shaft in the retracted configuration.

In accordance with another aspect of the disclosure, a surgical device includes a handle assembly, an elongate assembly detachably coupled to the handle assembly, and an end effector attachable to an adapter portion of the elongate assembly. The elongate assembly includes a telescopic shaft transitionable between a retracted configuration and an extended configuration to selectively adjust a length of the surgical device, and the adapter portion coupled to the telescopic shaft.

### BRIEF DESCRIPTION OF DRAWINGS

The above and other aspects and features of this disclosure will become more apparent in view of the following detailed description when taken in conjunction with the accompanying drawings wherein like reference numerals identify similar or identical elements.

FIG. 1 is a perspective view of a surgical device in accordance with this disclosure;

FIG. 2 is a perspective view of the surgical device of FIG. 1 with a telescopic shaft in an extended configuration;

FIG. 3 is a perspective view of the surgical device of FIG. 1 with the telescopic shaft in a retracted configuration, illustrating use with a cartridge; and

FIG. 4 is a side view of a handle assembly of the surgical device of FIG. 1, illustrating the handle assembly in a package.

### DETAILED DESCRIPTION

The surgical device disclosed herein is described in detail with reference to the drawings, in which like reference numerals designate identical or corresponding elements in each of the several views.

As used herein, the term “distal” refers to the portion that is being described which is further from a user, while the term “proximal” refers to the portion that is being described which is closer to a user. In addition, the terms parallel and perpendicular are understood to include relative configurations that are substantially parallel and substantially perpendicular up to about + or −10 degrees from true parallel and true perpendicular. Further, to the extent consistent, any or all of the aspects detailed herein may be used in conjunction with any or all of the other aspects detailed herein.

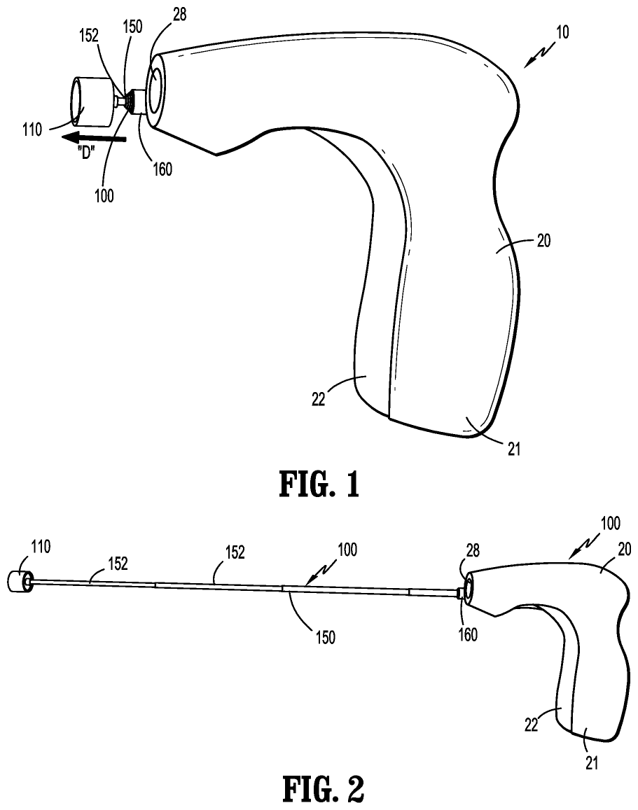

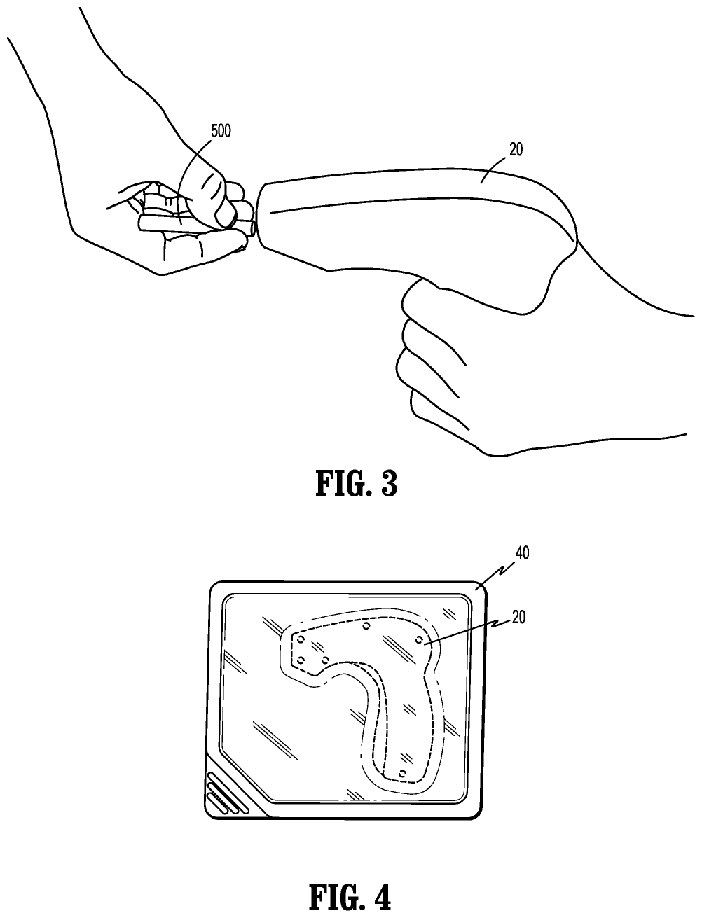

In FIGS. 1 and 2, an elongate assembly for use with a surgical device, in the form of an endoscopic surgical tack applier 10 is shown generally as 100. The endoscopic surgical tack applier 10 includes a handle assembly 20, the elongate assembly 100 extending from the handle assembly 20, and a cartridge 500 (FIG. 3) configured to store and selectively release or fire a plurality of tacks therefrom. It is contemplated that while the elongate assembly 100 is configured for use with the endoscopic surgical tack applier 10, the elongate assembly 100 may be configured for use with other surgical devices such as manual, mechanical and/or electromechanical surgical tack appliers, surgical clip appliers, surgical staplers, surgical stitching devices and the like.

The handle assembly 20 includes a handle housing 21 including a trigger 22 operatively coupled to the cartridge 500 (FIG. 3) such that actuation of the trigger 22 fires the plurality of tacks from the cartridge 500. In particular, the trigger 22 is operatively connected to a drive mechanism (not shown) as known by one skilled in the art such that each squeeze or actuation of the trigger 22 results in a rotation of an inner member assembly (not shown) of the cartridge 500 which, in turn, fires the tacks therefrom and into a surgical mesh (not shown) overlying tissue. For example, the drive mechanism may include, e.g., a flexible shaft, to impart rotation to the cartridge 500. The drive mechanism may be mechanically actuated or may include an electro-mechanical configuration including a motor to actuate firing of the tacks.

FIG. 1 illustrates the elongate assembly 100 detached from the handle assembly 20. However, it is further contemplated that the elongate assembly 100 may be fixedly coupled to the handle assembly 20. The handle assembly 20 defines an opening 28. The elongate assembly 100 includes an adapter portion 110, a telescopic shaft 150, and a supporting portion 160. In particular, the supporting portion 160 is configured to be detachably coupled to the handle assembly 20. It is envisioned that the handle assembly 20 may include a locking mechanism (not shown) configured to detachably fix the supporting portion 160 to the handle assembly 20. The telescopic shaft 150 extends from the supporting portion 160. The telescopic shaft 150 in the extended configuration may extend through the opening 28 of the handle assembly 20. The telescopic shaft 150 includes a plurality of partially overlapping segments 152 that are concentrically arranged. Each segment 152 of the plurality of overlapping segments 152 is configured to be displaced relative to the other segments 152 of the plurality of segments 152 to provide a variable length of the telescopic shaft 150. Each segment 152 may frictionally engage the adjacent segments 152 to maintain its relative position to the other segments 152. In this manner, the telescopic shaft 150 may maintain the desired length during the surgical procedure. The adapter portion 110 is configured to support the cartridge 500 therein. The adapter portion 110 may have a diameter greater than a diameter of the telescopic shaft 150.

The telescopic shaft 150 may be tapered along a length thereof to facilitate transition between the extended and retracted configurations. However, it is contemplated that the telescopic shaft 150 may have a substantially uniform diameter. Under such a configuration, the clinician may selectively adjust the length of the telescopic shaft 150. In this manner, the elongate assembly 100 may be transitionable between a retracted configuration (FIG. 1) and an extended configuration (FIG. 2). The telescopic shaft 150 defines a lumen (not shown) configured to receive, e.g., the drive mechanism, therethrough. For example, at least one of the adapter portion 110 or the telescopic shaft 150 may be formed of suitable biocompatible materials such as medical grade metals (e.g., stainless steel, titanium or aluminum). However, it is contemplated that other materials that do not cause substantial deflection of the telescopic shaft under a load in the extended configuration may be utilized.

In the retracted condition, the adapter portion 110 of the elongate assembly 100 is at least partially received in the opening 28 of the handle assembly 20. The plurality of overlapping segments 152 provides sufficient friction to maintain the desired length of the elongate assembly 100. By placing the elongate assembly 100 in the retracted configuration (FIG. 3), the clinician may easily load the cartridge 500 to the elongate assembly 100 while holding the handle assembly 20 compared to a conventional non-adjustable elongate shaft. In addition, by providing the adjustable elongate assembly 100, dimensions of a packaging 40 of the endoscopic surgical tack applier 10 are reduced, thereby saving space during transport and storage. For example, a length of the telescopic shaft 150 in the extended configuration may be more than twice the length of the telescopic shaft 150 in the retracted configuration.

The cartridge 500 may include at least an outer tube, a coil or helical thread provided along an interior of the outer tube, and an inner shaft rotatably disposed within the coil or helical thread as known by one skilled in the art. The inner shaft may include a splined distal end portion configured to support at least one anchor or tack, and a proximal end portion configured and adapted for mechanical and to a distal end of an intermediate flexible drive cable.

In use, the elongate assembly 100 is initially placed in the retracted configuration. The supporting portion 160 is coupled to the handle assembly 20 through the opening 28 of the handle assembly 20. The cartridge 500 may be loaded into the adapter portion 110. Thereafter, the telescopic shaft 150 is transitioned to the extended configuration based on the desired length of the telescopic shaft 150 by moving, e.g., the adapter portion 100, in the direction of an arrow “D” (FIG. 1). For example, the length of the telescopic shaft 150 may be tailored to the patient or the surgical procedure being performed. At this time, the cartridge 500 now attached to the adapter portion 110 may be positioned adjacent a target tissue through an opening or an incision. The endoscopic surgical tack applier 10 may be actuated to apply tacks to tissue and/or a surgical mesh as needed. Actuation of the trigger 22 results in a firing of a single tack from the cartridge 500. The endoscopic surgical tack applier 10 may be repeatedly fired to release tacks from the cartridge 500 until the surgical procedure is complete or until the cartridge 500 is out of tacks. The spent cartridge 500 may be replaced with a new cartridge 500 if additional tacks are needed to complete the surgical procedure.

It is also envisioned that the elongate assembly 100 may be configured to be connected to a robotic arm of a robotic surgical system to enable manipulation and control thereof.

While the disclosure has been shown in the drawings, it is not intended that the disclosure be limited thereto, as it is intended that the disclosure be as broad in scope as the art will allow and that the specification be read likewise. Therefore, the above description should not be construed as limiting, but merely as exemplifications. Those skilled in the art will envision other modifications within the scope and spirit of the claims appended hereto.

What is claimed is:

1. A surgical device comprising:
a handle assembly;
an elongate assembly detachably coupled to the handle assembly, the elongate assembly including:
a telescopic shaft transitionable between a retracted configuration and an extended configuration to selectively adjust a length of the surgical device; and
an adapter portion coupled to the telescopic shaft; and
a cartridge operatively coupled to the handle assembly and including a plurality of anchors such that actuation of the handle assembly fires an anchor from the cartridge.

2. The surgical device according to claim 1, wherein the telescopic shaft is tapered along a length thereof.

3. The surgical device according to claim 1, wherein the handle assembly defines an opening configured to receive the telescopic shaft therethrough.

4. The surgical device according to claim 1, wherein the entire telescopic shaft is received in the handle assembly when the telescopic shaft is in the retracted configuration.

5. The surgical device according to claim 1, wherein the telescopic shaft defines a lumen therethrough.

6. The surgical device according to claim 1, wherein the adapter portion is distal of the telescopic shaft.

7. The surgical device according to claim 1, wherein the adapter portion has a diameter larger than a diameter of a distal portion of the telescopic shaft.

8. The surgical device according to claim 1, wherein at least a portion of the adapter portion is received in the handle assembly when the telescopic shaft is in the retracted configuration.

9. The surgical device according to claim 1, wherein the telescopic shaft includes a plurality of segments concentrically arranged.

10. The surgical device according to claim 9, wherein each segment of the plurality of segments partially overlaps adjacent segments.

11. The surgical device according to claim 10, wherein each segment frictionally maintains a relative position thereof with respect to the adjacent segments.

12. The surgical device according to claim 1, wherein at least one of the telescopic shaft or the adapter portion is formed of a biocompatible material.

13. The surgical device according to claim 12, wherein the at least one of the telescopic shaft or the adapter portion is formed of a medical grade metal.

14. The surgical device according to claim 1, wherein the adapter portion of the elongate assembly is configured to support the cartridge therein.

15. The surgical device according to claim 1, wherein a length of the telescopic shaft in the extended configuration is more than twice the length of the telescopic shaft in the retracted configuration.

16. A surgical device comprising:
a handle assembly;
an elongate assembly detachably coupled to the handle assembly, the elongate assembly including:
a telescopic shaft transitionable between a retracted configuration and an extended configuration to selectively adjust a length of the surgical device; and
an adapter portion coupled to the telescopic shaft; and
an end effector attachable to the adapter portion of the elongate assembly.

17. The surgical device according to claim 16, wherein the entire telescopic shaft is received in the handle assembly when the telescopic shaft is in the retracted configuration.

18. The surgical device according to claim 16, wherein the adapter portion has a diameter larger than a diameter of a distal portion of the telescopic shaft.

19. The surgical device according to claim 16, wherein at least a portion of the adapter portion is received in the handle assembly when the telescopic shaft is in the retracted configuration.

20. The surgical device according to claim 16, wherein the telescopic shaft includes a plurality of segments concentrically arranged.
